(19) United States
(12) Patent Application Publication
(10) Pub. No.: US 2023/0303982 A1
(43) Pub. Date: Sep. 28, 2023

(54) CELLULOSE BINDING DOMAIN (CBD) CELL EFFECTOR PROTEIN (CEP) CHIMERA, FOR THE TISSUE ENGINEERING

(71) Applicants: BIOBETTER LTD., Kiryat Shmona (IL); Yissum Research Development Company of the Hebrew University of Jerusalem Ltd., Jerusalem (IL)

(72) Inventors: Oded Shoseyov, Karmei Yosef (IL); Amit Yaari, Kibbutz Ein Dor (IL); Tzvi Zvirin, Holon (IL); Shiri Baran Elmedvi, Kibbutz Shamir (IL); Efrat Avigad Laron, Kibbutz Gonen (IL); Yonatan Eran, Kidmat Tzvi (IL)

(21) Appl. No.: 18/040,810

(22) PCT Filed: Aug. 19, 2021
(86) PCT No.: PCT/IL2021/051017

§ 371 Date: Feb. 6, 2023

Related U.S. Application Data
(60) Provisional application No. 63/069,080, filed on Aug. 23, 2020.

(51) Int. Cl. C12N 5/071; C12N 5/077; C12N 15/82; C07K 14/50; C07K 14/495; C07K 14/485; C07K 14/475; C07K 14/49; C07K 14/54; A23L 13/00

(52) U.S. Cl. CPC C12N 5/0697; C12N 5/0658; C12N 5/0653; C12N 15/8257; C07K 14/50; C07K 14/495; C07K 14/485; C07K 14/475; C07K 14/49; C07K 14/5412; C07K 14/5437; C07K 14/4756; A23L 13/00; C12N 2509/00; C12N 2533/50; C12N 2533/78; C12N 2501/115; C07K 2319/00

(57) ABSTRACT

Disclosed is a chimeric polypeptide for use in-vitro tissue engineering, the polypeptide including cellulose binding domain (CBD), a cell effector protein (CEP), and a linker linking the CBD to the CEP, as well as systems and method utilizing same.

### FIELD OF INVENTION

The present disclosure is generally directed to novel growth inducing/manipulating scaffolds for production of fibrous and other non-isotropic tissues, in particular cultured meat. Specifically, the invention relates to recombinant cellulose binding domain (CBD) fused to a cell effector protein (CEP), the CEP bound to cellulose fiber scaffolds.

### BACKGROUND

Extracellular matrix (ECM), a three-dimensional network consisting of extracellular macromolecules, small molecules, effector proteins and minerals, is an important regulator of cellular growth, proliferation and differentiation. In addition, it contributes to tissue order and direction ensuring that a well-functioning tissue is generated. However, at this stage there are no available techniques allowing cost-effective mimicking of the ECM environment and of tissue generation in-vitro. Among the recent technologies in the field of tissue engineering, the use of scaffolds is a key component. However, the existing technologies lack the ability to provide an ECM-like environment. There are many types of scaffold including porous, fibrous, hydrogels, microspheres and acellular scaffolds, which while performing as support structures, do not enable a directional distribution of the cells; they require complicated manipulation for each type of cell; are associated with reduced cell viability; and at times generate acidic byproducts.

Growth factors play a crucial role in tissue engineering by directing the fate of cells and allowing the formation of tissues. However, growth factors suffer from a tendency to lose their bioactivity due to naturally occurring changes. Some strategies have been developed to overcome this disadvantage, including the use of specific materials like hydrogels (e.g. gelatin, alginate) and hydrophobic polymers (e.g. polycaprolactone); however short half-life and instability remain major obstacles of efficient tissue engineering.

There thus remains a need for a tissue engineering scaffold enabling efficient generation of well-ordered and functional tissue, truly mimicking tissue generation by nature.

### SUMMARY OF THE INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

According to some embodiments, there is provided a chimeric polypeptide, as well as system and method utilizing same, for in-vitro tissue engineering.

The herein disclosed chimeric polypeptide includes a carbohydrate-binding module (CBM), preferably a cellulose binding domain (CBD), a cell effector protein (CEP); and a linker linking the CBD to the CEP. The herein disclosed polypeptide is advantageously configured to provide a tailormade environment offering the required specificity, order, direction and functionality required create a desired tissue.

According to some embodiments, the linker may include a cleavage site characterized by enabling cleavage by a site-specific protease at a predetermined cleavage efficiency, such that when the polypeptide is exposed to the protease, a sustained release of the CEP from the CBD is obtained. This advantageously increases the half-life of the CEP, thereby increasing the efficiency of the tissue engineering system.

According to some embodiments, the chimeric protein is bound to or capable of binding to a fiber or layer of cellulose fibers serving as a scaffold for the tissue growth. Advantageously, more than one scaffold may be utilized, thereby enabling generation of complex tissue (i.e. tissues made of more than one type of cells, such as but not limited to tissue including both adipose and muscle tissues.

According to some embodiments, there is provided a chimeric polypeptide for use in in-vitro tissue engineering, the polypeptide comprising a carbohydrate-binding module (CBM); a cell effector protein (CEP); and a linker linking the CBM to the CEP, wherein the linker comprises a cleavage site characterized by enabling cleavage by a site-specific protease at a predetermined cleavage efficiency, such that when the polypeptide is exposed to the protease a sustained release of the CEP from the CBM is obtained.

According to some embodiments, CBM is a cellulose binding domain (CBD). According to some embodiments, the CBD has at least 70%, at least 80%, at least 85%, at least 90%, at least 95% or 100% sequence homology to the amino acid sequence set forth in SEQ ID NO: 1: (ELQLNLKVEFYNSQPSDTTNSINPQFKVTNTGSSAIDLSKLTLRYYYTVDGQKDQTFWC DHAAIIGSQGSYNGITSNVKGTFVKMSSSTNNADTYLEISFTGGTLEPGAHVQIQGRFAK NDWSQYTQSNDYSFKSASQFVEWDQVTAYLNGVLVWGKEPGGSVVPSTQPVTTPPAT TKPPATTIPPS). Each possibility is a separate embodiment.

According to some embodiments, the linker has a length of 5-50 amino acids, 5-25 amino acids, 5-20 amino acids, 10-25 amino acids or any other suitable length within the range of 5-50 amino acids. Each possibility is a separate embodiment.

According to some embodiments, the linker has at least 70%, at least 80%, at least 85%, at least 90%, at least 95% or 100% sequence homology to the amino acid sequence set forth in SEQ ID NO: 2: (GAGGGSGGGSGGGSAGGG). Each possibility is a separate embodiment.

According to some embodiments, the cleavage site may have the amino acid sequence set forth in SEQ ID NO: 3 (ENLYFQG) or SEQ ID NO 4: (ENLYFSG).

According to some embodiments, the cleavage site is positioned about 5 amino acids or less upstream of the N′ terminus of the CEP or about 5 amino acids or less downstream of the C′ terminus of the CEP. According to some embodiments the cleavage site may be contiguous to the linker. According to some embodiments, the amino acid encoding the linker-cleavage-site-peptide may have at least 70%, at least 80%, at least 85%, at least 90%, at least 95% or 100% sequence homology to the amino acid sequence set forth in SEQ ID NO: 5: (GAGGGSGGGSGGGSAGGGENLYFXG). Each possibility is a separate embodiment.

According to some embodiments, the amino acid encoding the linker-cleavage-site-CBD-peptide may have at least 70%, at least 80%, at least 85%, at least 90%, at least 95% or 100% sequence homology to the amino acid sequence set forth in SEQ ID NO: 6: (ELQLNLKVEFYNSQPSDTTNSINPQFKVTNTGSSAIDLSKLTLRYYYTVDGQKDQTFWC DHAAIIGSQGSYNGITSNVKGTFVKMSSSTNNADTYLEISFTGGTLEPGAHVQIQGRFAK NDWSQYTQSNDYSFKSASQFVEWDQVTAYLNGVLVWGKEPGGSVVPSTQPVTTPPAT TKPPATTIPPSGAGGGSGGGSGGGSAGGGENLYFXG), wherein X=Q or S. Each possibility is a separate embodiment.

According to some embodiments, the CEP is selected from: a growth factor, a hormone, a pro-apoptotic factor, an anti-apoptotic factor, a vascular growth factor, a cell differentiation factor, a bone growth factor, other protein required for cell viability like transferrin, or a combination thereof. Each possibility is a separate embodiment. According to some embodiments, the CEP is a cell differentiation factor, a growth factor or a growth hormone. Each possibility is a separate embodiment.

According to some embodiments, the CEP may be a cytokine or a growth factor selected from FGF2, IGF1, TGFβ1, EGF, LIF, Activin A, NRG1, PDGF, IL6, IL13 or any combination thereof. Each possibility is a separate embodiment.

According to some embodiments, the CEP may be an isoform of bovine FGF2 having at least 70%, at least 80%, at least 85%, at least 90%, at least 95% or 100% sequence homology to the amino acid sequence set forth in SEQ ID NO: 7: (PSLPEDGGSGAFPPGHFKDPKRLYCKNGGFFLRIHPDGRVDGVREKSDPHIKLQLQAEE RGVVSIKGVCANRYLAMKEDGRLLASKCVTDECFFFERLESNNYNTYRSRKYSSWYVA LKRTGQYKLGPKTGPGQKAILFLPMSAKS) Each possibility is a separate embodiment.

According to some embodiments, the chimeric polypeptide may further include an ER retention signal. According to some embodiments, the retention signal may have the amino acid sequence set for in SE ID NO: 8 (KDEL).

According to some embodiments, the chimeric polypeptide may include a sequence having at least 70%, at least 80%, at least 85%, at least 90%, at least 95% or 100% sequence homology to the amino acid sequence set forth in SEQ ID NO: 9: (ELQLNLKVEFYNSQPSDTTNSINPQFKVTNTGSSAIDLSKLTLRYYYTVDGQKDQTFWC DHAAIIGSQGSYNGITSNVKGTFVKMSSSTNNADTYLEISFTGGTLEPGAHVQIQGRFAK NDWSQYTQSNDYSFKSASQFVEWDQVTAYLNGVLVWGKEPGGSVVPSTQPVTTPPAT TKPPATTIPPSGAGGGSGGGSGGGSAGGGENLYFXGPSLPEDGGSGAFPPGHFKDPKRL YCKNGGFFLRIHPDGRVDGVREKSDPHIKLQLQAEERGVVSIKGVCANRYLAMKEDGR LLASKCVTDECFFFERLESNNYNTYRSRKYSSWYVALKRTGQYKLGPKTGPGQKAILFL PMSAKSKDEL), wherein X=Q or S. Each possibility is a separate embodiment.

According to some embodiments, upon cleavage the CEP may include a C-terminal glycine (remnant of the cleavage site) and the ER retention signal.

As a non-limiting example, after cleavage the chimeric polypeptide may include a sequence having at least 70%, at least 80%, at least 85%, at least 90%, at least 95% or 100% sequence homology to the amino acid sequence set forth in SEQ ID NO: 10 (GPSLPEDGGSGAFPPGHFKDPKRLYCKNGGFFLRIHPDGRVDGVREKSDPHIKLQLQAE ERGVVSIKGVCANRYLAMKEDGRLLASKCVTDECFFFERLESNNYNTYRSRKYSSWYV ALKRTGQYKLGPKTGPGQKAILFLPMSAKSKDEL).

According to some embodiments, the cleavage site is modified to provide a lower cleavage efficiency as compared to an unmodified version of the cleavage site. According to some embodiments, the modification comprises a deletion of one or more amino acids, an addition of one or more an amino acid, or a substitution of one or more amino acids. Each possibility is a separate embodiment. According to some embodiments, the modification comprises a chemical modification of one or more amino acids. As a non-limiting example, the glycine side chain at the P′1 position (the C terminus) of the TEV protease recognition site may be altered with little impact on the proteolytic efficiency DOI: 10.1016/S0006-291X(02)00574-0.

According to some embodiments, the cleavage site is a cleavage site of a proteas having a catalytic efficiency (k_{cat}/K_{M}) of below 1.5*10³ m⁻¹s⁻¹, of below 1*10³ m⁻¹s⁻¹, of below 0.5*10³ m⁻¹s^{31 1}, of below 1.5*10³ m⁻¹s⁻¹ or of below 15*10 m⁻¹s⁻¹ normal mammalian cell growth conditions. Each possibility is a separate embodiment.

According to some embodiments, the cleavage site is positioned such that the CEP is biologically active when released from the CBD. According to some embodiments, the cleavage site is positioned such that the CEP is only biologically active when released from the CBD. According to some embodiments, the CEP is only biologically active when released from the CBD, as a result of cleavage. According to some embodiments, the half-life of the CEP is higher when bound to CBD and/or to the linker as compared to in its free form. According to some embodiments, the linker increases the half-life of the CBD.

According to some embodiments, there is provided an in-vitro tissue engineering system comprising the polypeptide disclosed herein and at least one layer and/or fibers of cellulose.

According to some embodiments, the system includes at least two layers and/or fibers of cellulose. According to some embodiments, each of the at least two layers and/or fibers is associated with chimeric peptides including different CEPs.

According to some embodiments, the at least one layer comprises at least two types of chimeric polypeptides each comprising different CEPs. According to some embodiments, a first of the two types of chimeric polypeptides comprises a differentiation factor and a second of the two types of chimeric polypeptides comprises a growth factor associated with differentiation.

According to some embodiments, the linker of the first of the two types of chimeric polypeptides comprises a linker comprising a first cleavage site characterized by enabling cleavage by a site-specific protease at a first predetermined cleavage efficiency and the second of the two types of chimeric polypeptides comprises a linker comprising a second cleavage site characterized by enabling cleavage by a site-specific protease at a second predetermined cleavage efficiency, the second cleavage efficiency being lower than the first cleavage efficiency.

According to some embodiments, there is provided a vector comprising the herein disclosed chimeric polypeptide. According to some embodiments, the is a (pCAMBIA vector), as shown in FIG. 3.

According to some embodiments, there is provided a method for in-vitro tissue engineering, the method comprising:
a. proving cell growth medium comprising at least one layer and/or fiber of cellulose; the at least one layer of cellulose comprising a chimeric polypeptide comprising: a cellulose binding domain (CBD), a cell effector protein (CEP); and a linker linking the CBD to the CEP, wherein the linker comprises a cleavage site characterized by enabling cleavage by a site-specific protease;
b. seeding mammalian cells on the at least one layer of cellulose;
c. exposing the at least one layer of cellulose to the site-specific protease, such that the CEP is sustainably released into the cell growth medium
d. growing the cells until an organized tissue is obtained;
e. harvesting the tissue.

According to some embodiments, there is provided a method for in-vitro tissue engineering, the method comprising:
a. proving cell growth medium comprising at least one layer and/or fiber of cellulose; the at least one layer of cellulose comprising a chimeric polypeptide comprising: a cellulose binding domain (CBD), a cell effector protein (CEP); and a linker linking the CBD to the CEP,
b. seeding mammalian cells on the at least one layer of cellulose;
c. growing the cells until an organized tissue is obtained;
d. harvesting the tissue.

According to some embodiments, the at least one layer and/or fiber comprises a first layer with a first chimeric polypeptide comprising a first CEP and a second layer comprising a second chimeric polypeptide comprising a first CEP.

According to some embodiments, seeding the mammalian cells comprises seeding a first cell type on the first layer and a second cell type on the second layer. According to some embodiments, the first cell type comprises muscle cells and the second cell type comprises fat cells and wherein the organized tissue is meat. According to some embodiments, the first CEP is a muscle specific growth factor and the second CEP is an adipose specific growth factor.

According to some embodiments, the mammalian cells are multipotent or pluripotent cells and wherein the first and second factors cause differentiation of the cells into different cell types. According to some embodiments, the first CEP causes differentiation of the multipotent or pluripotent cells into muscle cells and the second CEP causes differentiation of the multipotent or pluripotent cells into fat cells.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions

### BRIEF DESCRIPTION OF THE DRAWINGS:

The invention will now be described in relation to certain examples and embodiments with reference to the following illustrative figures.

FIG. 1A is a schematic illustration of the herein disclosed chimeric polypeptide comprising a CEP (e.g. GF, hormone etc.—blue stared circle), fused a CBD by a linker which.

FIG. 1B is a schematic illustration of the herein disclosed chimeric polypeptide comprising a CEP (e.g. GF, hormone etc.—blue stared circle), fused a CBD by a linker chimeric polypeptide, the CBD bound to cellulose (gray sliver), which serves as a scaffold for tissue growth.

FIG. 2A is a schematic illustration of a multilayered cellulose scaffold including layers of cellulose, placed to provide a desired multilayered scaffold. Each layer may contain different chimeric polypeptides provide the suitable factors for differentiation and/or growth of different type of cells.

FIG. 2B is a schematic illustration of a multilayered cellulose scaffold with different layers of cellulose that serve as scaffolds for different cells. For example, muscle and fat cells for the creation of muscle and adipose tissue, respectively.

FIG. 2C is a schematic illustration of a tissue with desired characteristics (e.g. morphology, texture etc.), such as but not limited to cultured meat, obtained by differentiation and/or expansion of cells according to the CEP attached to the scaffold layers.

FIG. 3 is a map of the CBD-FGF2₁₄₆ₐₐ cassette inside the binary plasmid pCAMBIA.

FIG. 4 shows the level of cell viability as measured by a resazurin assay performed on MCF7 cells grown for 72 h under different growth conditions (medium supplemented with 10% FCS, FCS-free medium or FCS-free medium supplemented with a commercial FGF2 standard/146aa bovine FGF2 isoform/ CBD-FGF2. Results are shown as relative fluorescence units (RFU). *=p-value of <0.05 and **=p-value of <0.005.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances 10%, or in some instances ±5%, or in some instances ±1%, or in some instances ±0.1% from the specified value, as such variations are appropriate to perform the disclosed methods.

As used herein, the terms “polypeptide”, “peptide”, and “protein” may be used interchangeably and refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the invention can be produced by standard molecular biology techniques or by artificial synthesis and methods.

Variants of a particular polypeptide of the invention (i.e., the reference polynucleotide) can be evaluated by comparison of the percent sequence identity Thus, for example, polypeptide with a given percent sequence homology to the polypeptide of SEQ ID NO: 1 are disclosed. Percent sequence identity between any two polypeptides can be calculated using sequence alignment programs and parameters described elsewhere herein. For example, the polypeptide encoding the herein disclosed CBD may have a polypeptide with at least about 60%, 65%, 70%, 75%, 80%, 85%, 90%, 91%, 92%, 93%, 94%, 95%, 96%, 97%, 98%, 99% or more sequence identity across the entirety of the Seq ID NO:1.

Carbohydrate-binding modules (CBMs) are found in various different organisms. Cellulose Binding Domain (CBD) is one example of a CBMs. CBD is a protein domain isolated from the bacterium Clostridium cellulovorans or any other suitable genetic source (http://www.cazy.org/). It strongly binds to cellulose, and forms a stable, but reversible linkage that is able to withstand shear forces and mild changes in pH (pH 4 to 10) and salinity (10 mM to saturated NaCl). Linking CBD to a cell effector protein, such as but not limited to a growth factor (GF) or a hormone, increases the stability to the CEP. In addition, due to the size of the herein-disclosed chimeric polypeptide, the internalization rate of the CEP is slower, its half-life increased and thus lower working concentrations required. In addition, the presence of CBD links the CEP to the cellulose-based scaffold, which serves as a scaffold for the tissue growth and facilitates non-isotropic growth and development of the tissue, such as fibrous muscle for example or multi-directional tissue to give a desired 3D structure in another example.

According to some embodiments, the chimeric polypeptide includes a linker containing a cleavage site of a suitable protease. Advantageously, the linker itself is constructed such that it contributes to the conformational and proteolytic stability of the CEP. According to some embodiments, the protease may be a site-specific protease, an endogenous plant protease (Serine protease, Cysteine protease or other), a peptidase, and the like. Each possibility is a separate embodiment.

According to some embodiments, the protease cleavage site may be specific to a particular developmental stage of the grown tissue. According to some embodiments, cleavage-dependent release of the CEP advantageously ensures controlled release of the CEP into the culture media, thereby prolonging the half-life and thus require lower working concentrations.

According to some embodiments, the protease may be a TEV protease (EC 3.4.22.44, Tobacco Etch Virus nuclear-inclusion-a endopeptidase). Advantageously, TEV protease is a highly sequence-specific cysteine protease from Tobacco Etch Virus (TEV). According to some embodiments, the protease may be a furin protease with a minimal cleavage site of Arg-X-X-Arg. According to some embodiments, the protease may be a SUMO protease, which specifically recognize the tertiary sequence of the entire SUMO domain and cleaves off a double-glycine (GG) motif at its C-terminus of the recognition site. This process leaves no extra residues on the target proteins. Moreover, as SUMO proteases recognize the SUMO domain, they are more highly specific than other proteases. As used herein, the term “cell effector protein (CEP)” may refer to any factor affecting cell growth and/or differentiation, when present in the growth media of cells. According to some embodiments, the CEP may be a growth factor (for example TGF beta, FGF2), a hormone (e.g., insulin), a factor affecting vascularization of tissues or a factor inducing apoptosis etc. Each possibility is a separate embodiment.

As used herein, the term “chimeric” with referral to a polypeptide refers to a peptide joining amino-acids which originally encode separate proteins. Translation of this chimeric peptide results in a single polypeptide with functional properties derived from each of the original proteins.

Advantageously the herein disclosed chimeric polypeptide, and in-vitro tissue engineering system utilizing same, is versatile, i.e., various tissues may be engineered, based on the composition of the CEP(s), the concentration of the CEP(s), ratio between different chimeric molecules including different CEPs and their distribution on the cellulose scaffold.

According to some embodiment, the chimeric polypeptide may be expressed in a plant, such as but not limited to tobacco plants, genetically modified to express the chimeric polypeptide. Protein expression in tobacco plants carry great advantages over other expression systems. Among these advantages is the low cost of production, the potential for large-scale cultivation, and inherent safety reflecting the inability of human pathogens to replicate in plants. According to another embodiment, the chimeric polypeptide may be expressed in other plants such as soybean, and corn, for example. According to another embodiment, the chimeric polypeptide may be expressed in another organism, such as duckweed, yeast, bacteria, fungus, or algae, for example. Each possibility is a separate embodiment.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

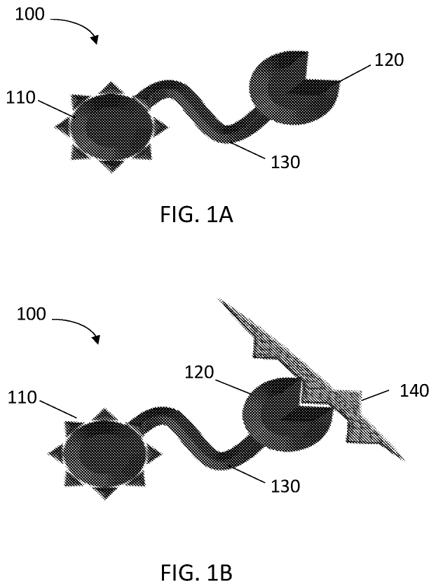

Reference is now made to FIG. 1A which is a schematic illustration of the hereindisclosed chimeric polypeptide 100 including a CEP 110 (e.g. GF, hormone etc.), fused to a CBD 120 by a linker 130, which contains a cleavage site of a site-specific protease. As seen from FIG. 1B, CBD 120 of chimeric polypeptide 100 is bound to or capable of binding to cellulose 140, which serves as a scaffold for the tissue growth.

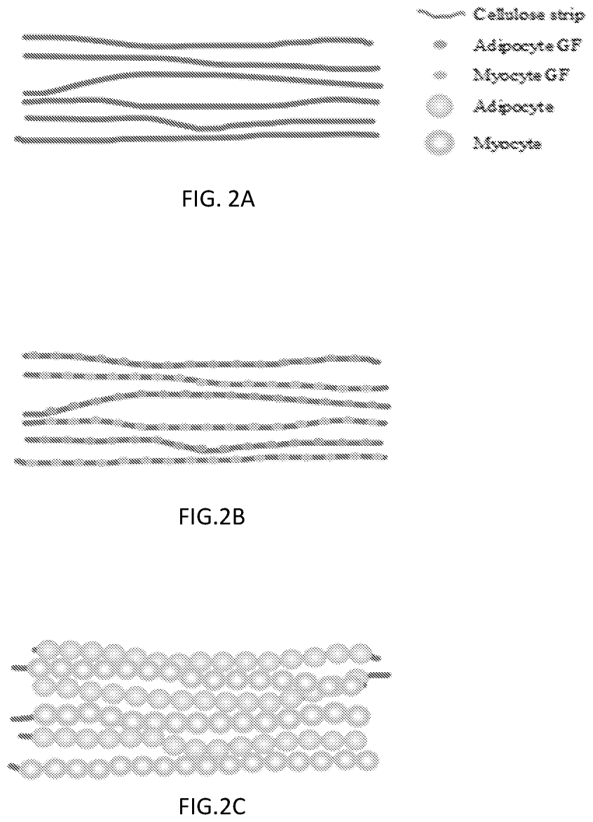

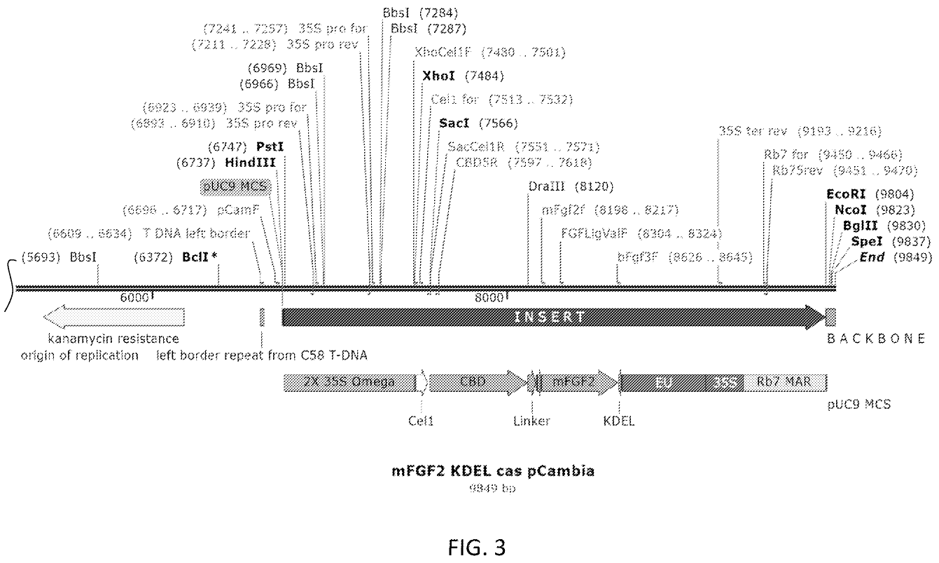

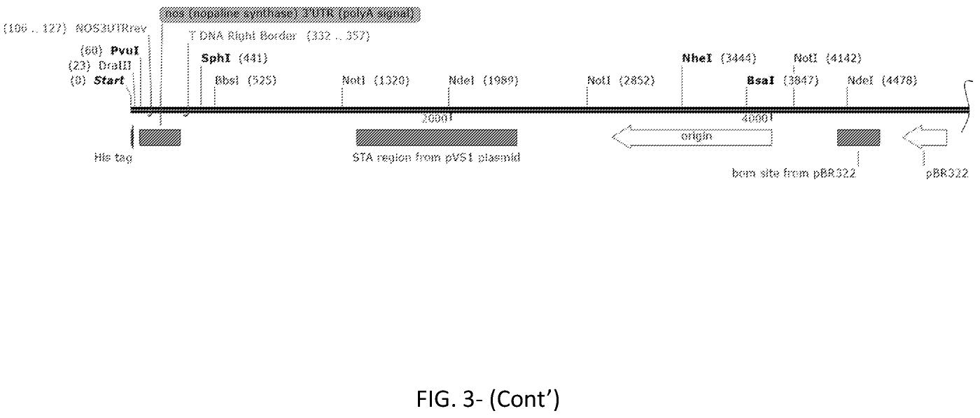

Reference is now made to FIG. 2A-FIG. 2C, which are schematic illustration of multilayered cellulose scaffolds. The scaffold may include layers of cellulose, placed to provide a desired multilayered scaffold, as seen in FIG. 2A. Each layer may contain chimeric polypeptides with different CEPs (e.g., adipocyte growth factor and myocyte growth factor) each allowing differentiation and/or growth of different types of cells (e.g., adipocytes and myocytes respectively), as shown in FIG. 2B. The cells may, as a result, differentiate and/or expand according to the CEP provided, thereby generating tissue with desired characteristics (e.g., morphology, texture etc.) such as but not limited to that of cultured meat (see FIG. 2C).

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1—Viability of Cells Grown in Starvation Media in the Presence of the herein Disclosed Polypeptide

In order to evaluate the activity of a CBD-bound growth factor, survival of cells grown in starvation medium and supplemented only with the herein disclosed chimeric polypeptide.

In short, MCF-7 cells were grown in a 96-well plate (10K cells per well) for three days in EMEM medium supplemented with 10% fetal craft serum (FCS) (positive control), in FCS-free EMEM (a complete starvation—negative control) or in starvation medium supplemented with increasing concentrations of a commercial recombinant human FGF2 standard (rhFGF2, Peprotech, USA #100-18b) FGF2, a 146aa bovine FGF2 isoform (seq ID NO: 6) or the 146aa bovine FGF2 isoform linked through a TEV cleavage-containing linker to CBD (CBD-FGF2 - set forth in SEQ ID NO: 10)

To assess the ability of plant-derived recombinant bovine FGF2 in retaining viability of MCF-7 cells during the 72 h starvation, a resazurin assay was conducted. The old medium was discarded and replaced with transparent DMEM medium (high glucose, no phenol red) containing resazurin (Catalog # ab129732, ABCAM, USA). The cells were incubated for 4 h at 37° C. and 5% CO₂, during which the fluorescence intensity (excitation: 535 nm, emission wavelength: 590 nm) was measured after 4 hours.

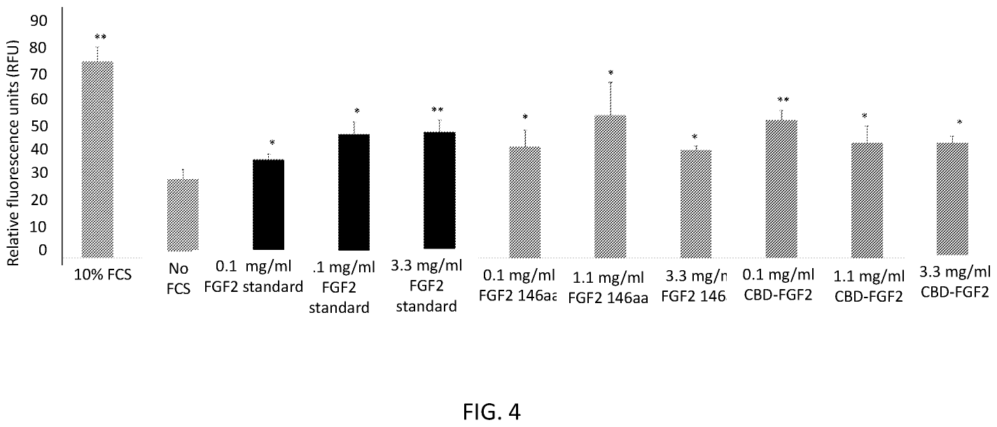

As seen from FIG. 4, whereas the cell viability dropped dramatically as a result of the 3-day starvation, as compared to the positive control (10% FCS), a significant rescue was observed if the cells were supplied with FGF2 (whether standard or 146aa bovine isoform). Importantly, a similar rescue was also obtained when the cells were supplied with the CBD-bound FGF2 indicating that CBD bound FGF2 can serve as a growth factor and that the CBD does not negatively influence survival.

### Example 2—Production of the Chimeric Polypeptide in Plants

Tobacco plants are transformed to express the hereindisclosed chimeric polypeptide and the plants are grown.

Once a sufficient biomass of plants is produced, the leaves of the plant are collected and the chimeric polypeptide extracted from the leaves by shredding and wringing. Following extraction, the homogenate is filtered on a 0.2 μm filter.

At this point, three different options are available.
1. The first option is to produce a cellulose fiber; each fiber is then linked to the chimeric polypeptide through the CBD. This way, a tailormade scaffold containing a desired CEP. Subsequently, cells are seeded on the scaffold to initiate tissue formation (e.g., meat) in a desired order and direction.
2. The second option is to pre-print the desired cellulose-chimeric polypeptide complex. In this case, the chimeric polypeptide chimera is used as cartridge (loading material). again, this application allows to control cultured tissue order and direction.
3. Finally, adding the chimeric polypeptide to a cell culture medium containing a cellulose-based scaffold.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

### Example 3—Testing Biological Activity of Cellulose-Bound FGF2

The purpose of this experiment is to demonstrate the biological activity of a cellulose-bound growth factor, namely bovine FGF2, by inducing attachment and growth of a human epithelial cell-line, MCF-7 in a starvation medium containing cellulose-bound growth factor.

In short 96-well Poly-L-Lysine plates are coated with Crystal Nano Cellulose (CNC) as the basis for cell attachment and growth that is later evaluated by a resazurin assay for cell viability. CNC coating allows a strong attachment to the plate bottom of target proteins fused to a cellulose-binding domain (CBD) via the CBD-cellulose interaction. To prepare a CNC coated plate, CNC is added and incubated at 25° C. for 10 minutes. Pre-diluted standard curve and samples of CBD fused-protein/s are added into the wells and incubated at 4° C. for 2 h, allowing any CBD-protein fusion protein to bind to the CNC. In this example, a CBD is linked to a 155 amino acid isoform of bovine FGF2.

After incubation the plate is washed (to remove any non-reactive components) with wash buffer and cells are immediately added to the plate for attachment and growth under various conditions: (a) serum-rich medium, (b) serum-free medium (“starvation condition”) and (c) serum-free medium supplied with CBD-FGF2. Resazurin cell viability fluorescent assay, which detects and quantify metabolic activity of cells, is performed after 24, 48, 72 and 96 hours, to determine the effect of CBD- FGF2 on cell attachment, growth and viability. In this assay, the blue non-fluorescent resazurin reagent is reduced to highly fluorescent resorufin by dehydrogenase enzymes in metabolically active cells. This conversion only occurs in viable cells and thus, the amount of resorufin produced is proportional to the number of viable cells in the sample. The resorufin formed in the assay can be quantified by measuring the relative fluorescence units (RFU) using a fluorometer (Ex=530-570 nm, Em=590-620 nm).

1-43. (canceled)

44. An in-vitro tissue engineering system comprising a chimeric polypeptide and at least one layer and/or fiber of cellulose, wherein the chimeric polypeptide comprises a cellulose binding domain (CBD), a cell effector protein (CEP); and a linker linking the CBD to the CEP.

45. The tissue engineering system of claim 44, wherein the linker has a length of 10-25 amino acids.

46. The tissue engineering system of claim 44, wherein the CEP is selected from the group consisting of a growth factor, a hormone, a cytokine, a pro-apoptotic factor, an anti-apoptotic factor, a vascular growth factor, a cell differentiation factor, a bone growth factor, other protein required for cell viability like transferrin, and a combination thereof.

47. The tissue engineering system of claim 46, wherein the CEP is selected from the group consisting of FGF2, IGF1, TGF1β, EGF, LIF, Activin A, NRG1, PDGF, IL6, IL13, and any combination thereof.

48. The tissue engineering system of claims 44, wherein the linker comprises a cleavage site characterized by enabling cleavage by a site-specific protease at a predetermined cleavage efficiency, such that when the chimeric polypeptide is exposed to the protease, a sustained release of the CEP from the CBD is obtained.

49. The tissue engineering system of claim 48, wherein the cleavage site is a cleavage site of a protease having a catalytic efficiency (k_{cat}/K_{M}) of below 1.5*10³ m⁻¹s⁻¹ at normal mammalian cell growth conditions.

50. The tissue engineering system of claim 44, comprising at least two layers and/or fibers of cellulose, wherein the at least one layer comprises at least two types of chimeric polypeptides, each comprising different CEPs.

51. The tissue engineering system of claim 50, wherein a first of the two types of chimeric polypeptides comprises a differentiation factor and a second of the two types of chimeric polypeptides comprises a growth factor associated with differentiation.

52. A chimeric polypeptide for use in in-vitro tissue engineering, the chimeric polypeptide comprising:
a. a cellulose binding domain (CBD);
b. a cell effector protein (CEP); and
c. a linker linking the CBD to the CEP.

53. The chimeric polypeptide of claim 52, wherein the linker has a length of 10-25 amino acids.

54. The chimeric polypeptide of claim 52, wherein the CEP is selected from the group consisting of a growth factor, a hormone, a cytokine, a pro-apoptotic factor, an anti-apoptotic factor, a vascular growth factor, a cell differentiation factor, a bone growth factor, other protein required for cell viability like transferrin, and a combination thereof.

55. The chimeric polypeptide of claim 52, wherein the CEP is selected from the group consisting of FGF2, IGF1, TGF1β, EGF, LIF, Activin A, NRG1, PDGF, IL6, IL13, and any combination thereof.

56. The chimeric polypeptide of claim 52, wherein the linker comprises a cleavage site characterized by enabling cleavage by a site-specific protease at a predetermined cleavage efficiency, such that when the chimeric polypeptide is exposed to the protease, a sustained release of the CEP from the CBD is obtained.

57. The chimeric polypeptide of claim 56, wherein the cleavage site is a cleavage site of a protease having a catalytic efficiency (k_{cat}/K_{M}) of below 1.5*10³ m⁻¹s⁻¹ at normal mammalian cell growth conditions.

58. A method for in-vitro tissue engineering, the method comprising:
a. providing a cell growth medium comprising at least one layer and/or fiber of cellulose, the at least one layer of cellulose comprising a chimeric polypeptide comprising a cellulose binding domain (CBD), a cell effector protein (CEP), and a linker linking the CBD to the CEP;
b. seeding mammalian cells on the at least one layer of cellulose;
c. growing the cells until an organized tissue is obtained; and
d. harvesting the tissue.

59. The method of claim 58, wherein the at least one layer and/or fiber comprises a first layer with a first chimeric polypeptide comprising a first CEP and a second layer comprising a second chimeric polypeptide comprising a second CEP.

60. The method of claim 59, wherein seeding the mammalian cells comprises seeding a first cell type on the first layer and a second cell type on the second layer.

61. The method of claim 60, wherein the first cell type comprises muscle cells and the second cell type comprises fat cells and wherein the organized tissue is meat and wherein the first CEP is a muscle specific growth factor and the second CEP is an adipose specific growth factor.

62. The method of claim 58, wherein the mammalian cells are multipotent or pluripotent cells and wherein the first and second factors cause differentiation of the cells into different cell types.

63. The method of claim 58, wherein the linker comprises a cleavage site characterized by enabling cleavage by a site-specific protease and wherein the method further comprises exposing the at least one layer of cellulose to the site-specific protease, such that the CEP is sustainably released into the cell growth medium.


SEQUENCE LISTING (10 sequences)

SEQ ID NO: 1 - protein, 187 aa, Artificial Sequence
  Synthetic peptide
ELQLNLKVEFYNSQPSDTTNSINPQFKVTNTGSSAIDLSKLTLRYYYTVDGQKDQTFWCDHAAIIGSQGSYNGITSNVKGTFVKMSSSTNNADTYLEISFTGGTLEPGAHVQIQGRFAKNDWSQYTQSNDYSFKSASQFVEWDQVTAYLNGVLVWGKEPGGSVVPSTQPVTTPPATTKPPATTIPPS

SEQ ID NO: 2 - protein, 18 aa, Artificial Sequence
  Synthetic peptide
GAGGGSGGGSGGGSAGGG

SEQ ID NO: 3 - protein, 7 aa, Artificial Sequence
  TEV cleavage site
ENLYFQG

SEQ ID NO: 4 - protein, 7 aa, Artificial Sequence
  TEV cleavage site
ENLYFSG

SEQ ID NO: 5 - protein, 25 aa, Artificial Sequence
  Synthetic peptide
  misc_feature (24)..(24): Xaa can be any naturally occurring amino acid
GAGGGSGGGSGGGSAGGGENLYFXG

SEQ ID NO: 6 - protein, 212 aa, Artificial Sequence
  Synthetic peptide
  misc_feature (211)..(211): Xaa can be any naturally occurring amino acid
ELQLNLKVEFYNSQPSDTTNSINPQFKVTNTGSSAIDLSKLTLRYYYTVDGQKDQTFWCDHAAIIGSQGSYNGITSNVKGTFVKMSSSTNNADTYLEISFTGGTLEPGAHVQIQGRFAKNDWSQYTQSNDYSFKSASQFVEWDQVTAYLNGVLVWGKEPGGSVVPSTQPVTTPPATTKPPATTIPPSGAGGGSGGGSGGGSAGGGENLYFXG

SEQ ID NO: 7 - protein, 146 aa, Artificial Sequence
  Synthetic peptide
PSLPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 8 - protein, 4 aa, Artificial Sequence
  ER retention signal
KDEL

SEQ ID NO: 9 - protein, 362 aa, Artificial Sequence
  Synthetic peptide
  misc_feature (211)..(211): Xaa can be any naturally occurring amino acid
ELQLNLKVEFYNSQPSDTTNSINPQFKVTNTGSSAIDLSKLTLRYYYTVDGQKDQTFWCDHAAIIGSQGSYNGITSNVKGTFVKMSSSTNNADTYLEISFTGGTLEPGAHVQIQGRFAKNDWSQYTQSNDYSFKSASQFVEWDQVTAYLNGVLVWGKEPGGSVVPSTQPVTTPPATTKPPATTIPPSGAGGGSGGGSGGGSAGGGENLYFXGPSLPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKSKDEL

SEQ ID NO: 10 - protein, 151 aa, Artificial Sequence
  Synthetic peptide
GPSLPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKSKDEL